(19) United States
(12) Patent Application Publication
(10) Pub. No.: US 2009/0162864 A1
(43) Pub. Date: Jun. 25, 2009

(54) BIOLOGICAL SUBSTANCE DETECTION CARTRIDGE,BIOLOGICAL SUBSTANCE DETECTION APPARATUS, AND BIOLOGICAL SUBSTANCE DETECTION METHOD

(75) Inventors: Rie KITAZAWA, Shiojiri (JP); Fumio TAKAGI, Chino (JP)

Correspondence Address: HARNESS, DICKEY & PIERCE, P.L.C., P.O. BOX 828, BLOOMFIELD HILLS, MI, 48303, US

(73) Assignee: SEIKO EPSON CORPORATION, Tokyo (JP)

(21) Appl. No.: 12/332,857

(22) Filed: Dec. 11, 2008

(30) Foreign Application Priority Data
Dec. 20, 2007 (JP) 2007-328434

(51) Int. Cl. C12Q 1/68; C12M 1/00

(52) U.S. Cl. 435/6; 435/287.2

(57) ABSTRACT

A biological substance detection cartridge, including: a reaction vessel for reacting a probe with a specific biological substance included in a sample solution, the reaction vessel having a region for fixing the probe for detecting the biological substance; a porous membrane facing the inside of the reaction vessel; a gas-liquid separation membrane superposed with the porous membrane; and a air discharge component which is provided on the opposite side of the gas-liquid separation membrane from the side contacting the porous membrane, and with which the interior can be kept at negative pressure during the reaction between the biological substance and the probe.

### CROSS-REFERENCES TO RELATED APPLICATIONS

This application relates to and claims priority from Japanese Patent Application No. 2007-328434, filed on Dec. 20, 2007, the entire disclosure of which is incorporated herein by reference.

### BACKGROUND

1. Technical Field

The present invention relates to a biological substance detection cartridge, biological substance detection apparatus, and biological substance detection method for detecting a biological substance, such as a nucleic acid molecule having a specific base sequence.

2. Related Art

A DNA microarray is one method for assaying whether or not a specific gene originating in a disease is present in a specimen such as blood or tissue cells. With a DNA microarray, the presence of a target gene is detected by reacting (hybridizing) a probe gene affixed to a plate with a gene in a specimen. In the past, attempts have been made at raising reaction efficiency between the probe gene and the specific gene in the specimen in order to improve accuracy in the detection of the specific gene included in the specimen.

For instance, Japanese Patent No. 3,746,756 discloses a method in which the space between a plate member and the plate to which a probe has been affixed is filled with a sample solution, and the plate and the plate member are moved relative to each other to agitate the sample solution and improve the reaction efficiency.

Japanese Patent No. 3,557,419 discloses a method in which reaction efficiency is improved by dispersing microparticles in the sample solution and agitating.

With the methods disclosed in Japanese Patent Nos. 3,746,756 and 3,557,419, the sample solution is agitated by rotating the DNA microarray, but JP-A-2007-40969 discloses, as an example of a method for raising reaction efficiency without using a mechanism for moving the microarray, a biochemical reaction cassette equipped with a fluid resistor that reduces the channel cross sectional area so as to control the flow of fluid within the chamber used for reacting the sample with the probe for detecting nucleic acid.

When a sample solution is agitated or a flow is brought about within a chamber as with the prior art disclosed in Japanese Patent Nos. 3,746,756 and 3,557,419 and in JP-A-2007-40969, bubbles tend to be generated within the sample solution. If bubbles are generated, they can impede contact between the probe gene and the gene in the specimen, which is a problem in that the reaction is uneven and inefficient.

### SUMMARY

In view of this, it is an object of the present invention to obtain a biological substance detection cartridge, biological substance detection apparatus, and biological substance detection method with which the reaction in the reaction vessel is prevented from becoming uneven, and reaction efficiency and detection sensitivity are higher.

The biological substance detection cartridge pertaining to the present invention comprises a reaction vessel for reacting a probe with a specific biological substance included in a sample solution, the reaction vessel having a region for fixing the probe for detecting the biological substance, a porous membrane facing the inside of the reaction vessel, a gas-liquid separation membrane superposed with the porous membrane, and a air discharge component which is provided on the opposite side of the gas-liquid separation membrane from the side contacting the porous membrane, and with which the interior can be kept at negative pressure during the reaction between the biological substance and the probe.

With the present invention, even if bubbles should be generated in the reaction vessel during the reaction, they can be discharged through the gas-liquid separation membrane, which prevents unevenness of the reaction in the reaction vessel, and raises both reaction efficiency and detection sensitivity. Also, by providing the porous membrane between the gas-liquid separation membrane and the reaction vessel, the probe can be fixed not only to the inner walls of the reaction vessel, but also on the porous membrane side.

It is preferable if the reaction vessel comprises a plurality of chambers for reacting the biological substance and the probe, each having a region for fixing the probe, and a channel provided between the plurality of chambers, the channels being such that the surface area of a cross section perpendicular to a direction in which the sample solution moves is smaller than the cross sectional area of the chambers.

As a result, different types of probes are each fixed in each of the various chambers linked by the channel, which allows a plurality of types of target to be detected all at once. Also, if just one type of probe is used in one chamber, even if the detection of the reaction result is performed using a chemiluminescent substance with which a luminescent substance floats up in the solution, there will be no problem with the luminescent substances becoming mixed so that it is impossible to match a reaction result with a probe.

Furthermore, since the surface area of a cross section perpendicular to the direction in which the sample solution moves is smaller than the cross sectional area of the chamber, the sample solution will flow from a channel with a small cross sectional area into a large chamber, which changes the flow of the liquid and has the effect of agitating the sample solution in the chamber. Agitating the sample solution in the chamber further increases reaction efficiency because more of the biological substance that is the target will come into contact with the probe in a shorter time.

The region for fixing the probe may be provided to the inner walls of the reaction vessel, or may be provided over the porous membrane. It may also be provided to both the inner walls and the porous membrane.

Providing the region for fixing the probe to both the inner walls and the porous membrane increases the surface area over which the biological substance that is the target comes into contact with the probe, which enhances reaction efficiency and detection sensitivity. Also, because the porous membrane has a three-dimensional structure, more probe can be fixed than to the inner walls of the reaction vessel, so reaction efficiency and detection sensitivity are improved.

Also, a plate having a through-hole corresponding to the region for fixing the probe may be provided between the reaction vessel and the porous membrane.

This allows the regions on the porous membrane where the probe is fixed to be separated from one another, and when different probes are fixed in adjacent fixing regions, it eliminates the problem of mixing of the probes that would make it impossible to tell which probe the reaction result came from.

Preferably, the reaction vessel is formed with a transparent plate.

This allows the interior of the reaction vessel to be observed from the outside, so the reaction and detection processing can be performed with the same apparatus, which affords a more compact apparatus and more efficient processing.

The biological substance detection apparatus pertaining to the present invention uses the above-mentioned biological substance detection cartridge to perform biological substance detection, and is equipped with a first pump for keeping the air discharge component at negative pressure during the reaction between the biological substance and the probe.

This allows any bubbles generated in the reaction vessel during the reaction to be discharged by a simple method through the gas-liquid separation membrane.

Also, it is preferable if a second pump is provided for reciprocally moving the sample solution within the reaction vessel.

This allows more of the biological substance that is the target to come into contact with the probe, so reaction efficiency is improved.

The biological substance detection method pertaining to the present invention involves the use of the above-mentioned biological substance detection apparatus, comprising a reaction step of supplying a sample solution into the reaction vessel, and reacting a specific biological substance included in the sample solution with a probe that is fixed in the reaction vessel and is used to detect the biological substance, and a detection step of detecting the biological substance reacted with the probe, wherein the air discharge component is kept at negative pressure in the reaction step, so that any bubbles in the reaction vessel are discharged through the gas-liquid separation membrane to the outside.

With the present invention, even if bubbles should be generated in the reaction vessel during the reaction, they can be discharged through a gas-liquid separation membrane, which prevents unevenness in the reaction inside the reaction vessel, and allows reaction efficiency and detection sensitivity to be increased.

Also, it is preferable if, in the detection step, detection of the biological substance reacted with the probe is performed by a method using a chemiluminescent substance.

In general, with a method in which a chemiluminescent substance is used, the amount of luminescent substance that is produced can be increased by increasing the amount of substrate that is added, so it is easy to raise the detection sensitivity.

### BRIEF DESCRIPTION OF THE DRAWINGS

FIG. 1 is an oblique view of the simplified configuration of a nucleic acid detection apparatus pertaining to Embodiment 1 of the present invention;

FIG. 2A is an exploded oblique view of the detection cartridge pertaining to Embodiment 1 of the present invention, and FIG. 2B is a cross section along the C-C line in FIG. 2A;

FIG. 3A, FIG. 3B, and FIG. 3C consist of diagrams of the pattern in which the probe fixing region is formed in the chambers;

FIG. 4A illustrates the principle of a detection method in which a chemiluminescent substance is used, and FIG. 4B illustrates the principle of a detection method in which a fluorescent labeling reagent is used;

FIG. 5A is an exploded oblique view of a detection cartridge pertaining to Embodiment 2 of the present invention, and FIG. 5B is a cross section along the C-C line in FIG. 5A;

FIG. 6A and FIG. 6B consist of diagrams of the pattern in which the probe fixing region is formed in the chambers;

FIG. 7A is an exploded oblique view of a detection cartridge pertaining to a variation of Embodiment 2 of the present invention, and FIG. 7B is a cross section along the C-C line in FIG. 7A; and

FIG. 8 is a diagram of the pattern in which the probe fixing region is formed in the chambers.

### DESCRIPTION OF EXEMPLARY EMBODIMENTS

Embodiments of the present invention will now be described through reference to the drawings.

### Embodiment 1

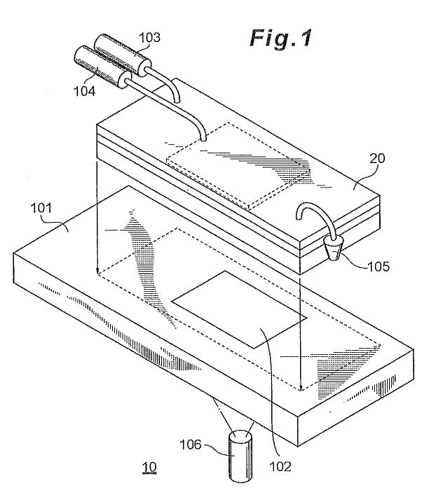

FIG. 1 is an oblique view of the simplified configuration of a nucleic acid detection apparatus (biological substance detection cartridge) 10 pertaining to Embodiment 1 of the present invention. As shown in the drawing, the nucleic acid detection apparatus 10 comprises a stage 101 on which is placed a detection cartridge (biological substance detection cartridge) 20, a detection window 102, a pump 103, a pump 104, a sample vessel 105, and a CCD camera 106.

The stage 101 is used to fix the detection cartridge 20. The detection window 102 is provided to the stage 101, and in detection in a hybridization reaction, the CCD camera 106 is used to measure the luminance intensity of the chemiluminescent substance produced in the detection cartridge 20.

The pumps 103 and 104 can be syringe pumps or micro-pumps, for example. The pump 103 is used to send the sample solution back and forth within the detection cartridge 20, while the pump 104 is used to discharge bubbles in the detection cartridge 20. The pumps 103 and 104 are connected to the detection cartridge 20 via capillary tubes composed of a fluororesin, a polyether ketone (PEEK) resin, a silicone resin, or the like.

The sample vessel 105 is a vessel for holding a specimen sample solution. The sample vessel 105 connected to the detection cartridge 20 via capillary tubes composed of a fluororesin, a polyether ketone (PEEK) resin, a silicone resin, or the like, and the sample solution is supplied from the sample vessel 105 into the detection cartridge 20. Any sample solution that overflows from the detection cartridge 20 in the course of the sample solution being pumped back and forth by the pump 103 first flows into the sample vessel 105, and then returns to the detection cartridge 20.

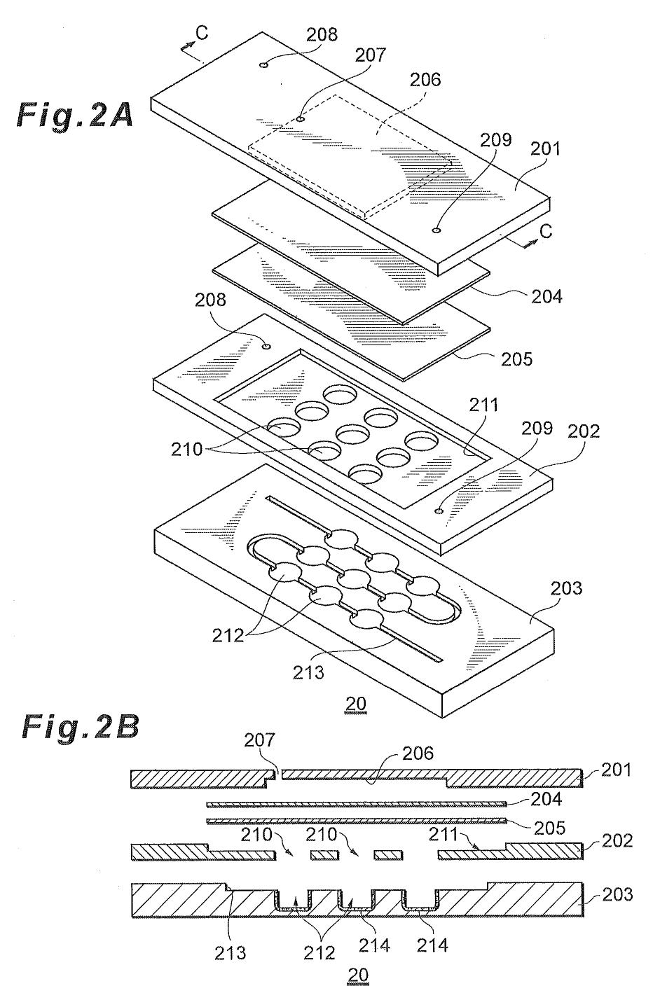

FIG. 2A is an exploded oblique view of the detection cartridge 20 pertaining to Embodiment 1 of the present invention, and FIG. 2B is a cross section along the C-C line in FIG. 2A. As shown in the drawings, the detection cartridge 20 is produced by sticking together a plate 201, a gas-liquid separation membrane 204, a porous membrane 205, a plate 202, and a plate 203.

A air discharge component 206 is formed in the plate 201. A discharge opening 207 is provided to the air discharge component 206, and the discharge opening 207 and the pump 104 are linked via a capillary tube. Also, the plate 201 is provided with liquid introduction ports 208 and 209 at locations corresponding to both ends of a channel 213 formed in the plate 203. The liquid introduction ports 208 and 209 are linked with the pump 103 and the sample vessel 105, respectively, via capillary tubes.

The gas-liquid separation membrane 204 and the porous membrane 205 are flanked by the plate 201 and the plate 202. The gas-liquid separation membrane 204 is a membrane formed from polytetrafluoroethylene resin or the like, and has the property of transmitting gas but blocking the permeation of liquids. The porous membrane 205 is formed from nitrocellulose, nylon, polycarbonate, or the like.

The plate 202 is provided with through-holes 210 at locations corresponding to chambers 212 formed in the plate 203. A membrane disposition recess 211 is formed for disposing the porous membrane 205 and the gas-liquid separation membrane 204. Just as with the plate 201, liquid introduction ports 208 and 209 are provided at locations corresponding to both ends of the channel 213 formed in the plate 203.

In the plate 203 are formed a plurality of the chambers 212 and the channel 213 that is provided so as to link the chambers 212. The two ends of the channel 213 are connected to the pump 103 and the sample vessel 105 via the liquid introduction ports 208 and 209, respectively. The plate 203 is preferably a transparent plate, such as a glass plate. This allows the interior of the chambers 212 to be observed from the outside, so the reaction and detection processing can be performed with the same apparatus.

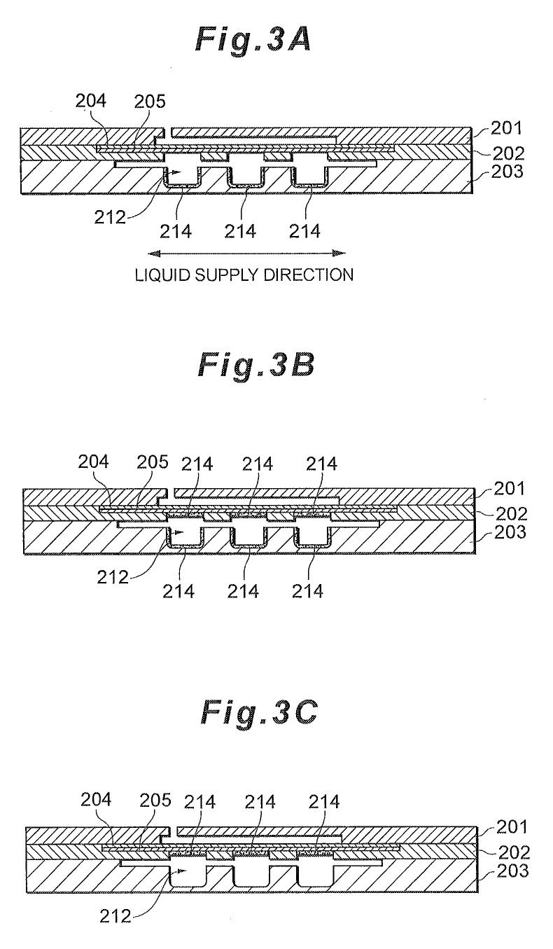

As shown in FIG. 3A, disposing the plate 203, the plate 202, and the porous membrane 205 superposed over one another results in the top being covered by the porous membrane 205, and a plurality of the linked chambers 212 being formed via the channel 213. Further, by superposing the gas-liquid separation membrane 204 and the plate 201 over this, a configuration is achieved with which any air in the chambers 212 is discharged by the air discharge component 206 through the gas-liquid separation membrane 204. Bubbles generated in the chambers 212 can be removed by keeping the air discharge component 206 at negative pressure.

The chambers 212 have, for example, a length of 200 μm in the liquid pumping direction, a width of 200 μm at a cross section perpendicular to the liquid pumping direction, and a depth of 150 to 200 μm. The channel 213 that links the chambers 212 together can have a length of 200 μm in the liquid pumping direction, a width of 100 μm at a cross section perpendicular to the liquid pumping direction, and a depth of 50 to 100 μm. The channel 213 is formed so that the surface area of a cross section perpendicular to the liquid pumping direction is smaller than that of a cross section of the chambers 212 perpendicular to the liquid pumping direction. The shape of the chambers 212 may be circular, or may be elliptical, quadrangular with rounded corners, or another such shape, but a shape with which bubbles are less likely to accumulate in the chambers 212 is preferable.

The chambers 212 each have a probe fixing region 214 on the inner walls. The probe fixing region 214 is a region coated with a probe. As shown in FIG. 3A, the probe fixing region 214 may be provided to the surface of the inner walls of the chambers 212, or as shown in FIG. 3B, may be provided to the surface of the porous membrane 205 and the inner walls of the chambers 212. With the configuration shown in FIG. 3B, the amount of probe in the chambers 212 increases, and the surface area over which the biological substance that is the target comes into contact with the probe is also greater, so reaction efficiency and detection sensitivity are enhanced. Also, as shown in FIG. 3C, the probe fixing region 214 may be provided only to the surface of the porous membrane 205. Since the porous membrane 205 has a three-dimensional structure, more probe can be fixed to it than to the inner walls of the chambers 212, so reaction efficiency and detection sensitivity can be enhanced.

The probe can be any substance capable of trapping the target substance included in the specimen sample, such as blood, urine, saliva, or spinal fluid. For example, if the target is a nucleic acid such as DNA or RNA, the probe can be a nucleic acid or nucleotide (oligonucleotide) or the like that will hybridize (complementarily bind) with these nucleic acids. For example, cDNA, a PCR product, or the like can be used as the nucleic acid.

The target is not limited to being a nucleic acid, though, and may be a specific protein, for example. In this case, the probe can be a substance capable of specifically trapping (adsorbing, binding, etc.) this protein. More specifically, examples include antigens, antibodies, receptors, enzymes, and other such proteins, peptides (oligopeptides), and so forth.

Coating the probe fixing regions 214 with the probe can be accomplished using a contact or non-contact type of spotter or the like. A non-contact spotter is preferably used to coat the porous membrane 205. In this embodiment, different types of probes are each fixed in each of the chambers 212. This allows a plurality of types of target to be detected all at once.

The probe fixing region 214 may be subjected to a surface treatment as needed. Examples of surface treatment include a treatment for securely fixing the probe to the surface of the probe fixing region 214 (solid-phase processing).

Next, we will describe the hybridization processing between the target (nucleic acid) and the probe (reaction step) and the hybridization detection processing (detection step) using the nucleic acid detection apparatus 10 pertaining to this embodiment.

First, the pump 103 is used to fill the detection cartridge 20 in which probes have been fixed to the probe fixing regions 214 (the space formed by the chambers 212 and the channel 213) with blocking buffer.

The pump 104 is used to put the interior of the air discharge component 206 under negative pressure, after which the pump 103 is used to pump the blocking buffer back and forth in the detection cartridge 20, thereby blocking the region where no probe has been fixed. This blocking is carried out for about 10 minutes.

Next, the pump 103 is used to discharge the blocking buffer, after which the pump 103 is used to fill the detection cartridge 20 with a detergent solution and to pump the detergent solution back and forth in the detection cartridge 20, so that the insides of the chambers 212 and the channel 213 are thoroughly cleaned.

Nest, the detection cartridge 20 is filled with a biotin-labeled sample solution. More specifically, the pump 103 is driven so that the sample solution held in the sample vessel 105 is supplied into the detection cartridge 20.

The method for preparing the biotin-labeled sample solution will be described now.

The sample solution includes biological samples such as blood, urine, saliva, and spinal fluid. The nucleic acid that is the target may be subjected to amplification by PCR as needed.

More specifically, first and second primers are added to the sample and a cycle that has three temperature steps is performed. The first primer specifically binds to part of the nucleic acid that is the target, and the second primer specifically binds to part of the nucleic acid that is complementary with the target nucleic acid. When the first and second primers bind to a double-stranded nucleic acid including the target nucleic acid, the double-stranded nucleic acid including the target nucleic acid is amplified by an extension reaction. After the double-stranded nucleic acid including the target nucleic acid has been sufficiently amplified, a third primer is added to the sample and a cycle that has three temperature steps is performed. The third primer is capable of incorporating biotin during the extension reaction, and specifically binds to part of the nucleic acid that is complementary with the target nucleic acid. When the nucleic acid that is complementary with the target nucleic acid binds to the third primer, the target nucleic acid labeled with biotin is amplified by an extension reaction. As a result, when the sample includes the target nucleic acid, a labeled target nucleic acid is produced, and when the sample does not include the target nucleic acid, a labeled target nucleic acid is not produced. Biotin was used here as the target substance, but it may instead be another enzyme, or a luminescent substance or the like.

Next, the biotin-labeled sample solution is pumped back and forth inside the detection cartridge 20 and reacted (hybridized) with the probe fixed to the probe fixing region 214. This hybridization is preferably carried out for 1 to 3 hours.

The pump 104 is used to keep the inside of the air discharge component 206 under negative pressure while hybridization is being performed, as well.

The detection cartridge 20 pertaining to this embodiment is formed such that the surface area of a cross section of the channel 213 perpendicular to the direction in which the sample solution flows is smaller than the cross sectional area of the chambers 212. When the sample solution flows from the channel 213 with a small cross sectional area into the larger chambers 212, this changes the flow of the liquid and has the effect of agitating the sample solution in the chambers 212. Agitating the sample solution in the chambers 212 increases hybridization efficiency because more of the target nucleic acid will come into contact with the probe in the probe fixing region 214 in a shorter time. On the other hand, since a liquid is readily agitated by turbulence, bubbles tend to accumulate in the chambers 212, and this can bring about an uneven hybridization reaction. With this embodiment, however, since the bubbles in the chambers 212 are discharged to the air discharge component 206 through the gas-liquid separation membrane 204 by using the pump 104 to keep the inside of the air discharge component 206 under negative pressure, unevenness of the reaction is prevented, and reaction efficiency and detection sensitivity can be improved.

Then, the pump 103 is used to discharge the biotin-labeled sample solution, after which the pump 103 is used to fill the detection cartridge 20 with detergent solution and to pump the detergent solution back and forth in the detection cartridge 20 to thoroughly clean the insides of the chambers 212 and the channel 213.

Next, the pump 103 is used to fill the detection cartridge 20 with a streptavidin-horseradish peroxidase (HRP) and to pump the solution back and forth for about 5 minutes in the detection cartridge 20.

The HRP solution is then discharged, after which the detection cartridge 20 is filled with a detergent solution, and the detergent solution is pumped back and forth in the detection cartridge 20 to thoroughly clean the insides of the chambers 212 and the channel 213.

Next, the pump 103 is used to fill the detection cartridge 20 with a solution containing hydrogen peroxide and a chemiluminescent substrate (luminol). Once filled, the inside of the air discharge component 206 returned to atmospheric pressure and allowed to stand for about 10 to 30 seconds without the liquid being pumped back and forth by the pump 103, and the production of the chemiluminescent substance is awaited.

Once the chemiluminescent substance has been produced, the CCD camera 106 is used to measure the luminance intensity to check whether a hybridization reaction has occurred.

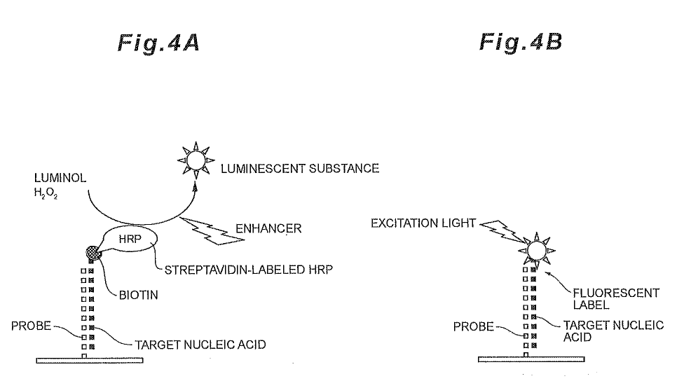

FIG. 4A illustrates the principle of a detection method in which a chemiluminescent substance is used. As shown in FIG. 4A, with a detection method in which a chemiluminescent substance is used, a streptavidin-horseradish peroxidase (HRP) that has been labeled with bound biotin and streptavidin is bound to the target nucleic acid, and a chemiluminescent substrate liquid (luminol and hydrogen peroxide) is added to this, the result being that the HRP reacts with the luminol and hydrogen peroxide, produces a luminescent substance, and thereby emits light. The amount of luminescent substance produced can be increased by increasing the luminol and hydrogen peroxide, so raising the detection sensitivity is easy.

FIG. 4B illustrates the principle of a detection method in which a fluorescent labeling reagent is used. With a method in which a fluorescent labeling reagent is used, a fluorescent labeling reagent bound to the target nucleic acid is irradiated with excitation light, whereupon it emits light. The luminance intensity is a function of the amount of fluorescent labeling reagent bound to the target nucleic acid, which means that raising the detection sensitivity is more difficult than with a detection method in which a chemiluminescent substance is used.

Therefore, a detection method in which a chemiluminescent substance is used is better in terms of raising detection sensitivity. Furthermore, with a method in which a fluorescent labeling reagent is used, since the fluorescent labeling reagent (a fluorescent substance) is in a state of being bound to the target nucleic acid, the position of the fluorescent substance does not move. Accordingly, even when hybridization is performed using a plurality of probes in a single chamber, it will be easy to distinguish the reaction results among the probes. On the other hand, with a method in which a chemiluminescent substance is used, the produced luminescent substances would end up being mixed in a single chamber, so when a plurality of probes are used in a single chamber, it will be impossible to tell what is detected by which probe. However, with the nucleic acid detection apparatus 10 pertaining to this embodiment, different types of probes are each fixed in each of the chambers 212. This means that even with a method in which a chemiluminescent substance is used, there will be no problem with the luminescent substances becoming admixed so that it is impossible to match a reaction result with a probe, and a plurality of kinds of target can be detected all at once. Furthermore, the enzyme, substrate, and so forth used in detection with a chemiluminescent substance are not limited to the examples given above.

As discussed above, with Embodiment 1, different types of probes are each fixed in each of the chambers 212 linked by the channel 213, which allows a plurality of kinds of target to be detected all at once. Also, if just one kind of probe is used in a single chamber, even when the detection of hybridization results is performed with a method in which a chemiluminescent substance is used, there will be no problem with the luminescent substances becoming mixed so that it is impossible to match a reaction result with a probe.

Furthermore, with this embodiment, since the surface area of a cross section of the channel 213 perpendicular to the direction in which the sample solution moves is smaller than the cross sectional area of the chambers 212, there will be a change in the flow at the boundary between the channel 213 and the chambers 212, which has the effect of agitating the sample solution in the chambers 212. Agitating the sample solution in the chambers 212 increases reaction efficiency because more of the target will come into contact with the probe in a shorter time.

Also, with this embodiment, since the pump 103 is used to send the sample solution back and forth within the chambers 212 and the channel 213, more of the target will come into contact with the probe, and this increases reaction efficiency.

Meanwhile, with this embodiment, turbulence is generated at the boundary between the channel 213 and the chambers 212, and bubbles tend to accumulate in the chambers 212, but by using the pump 104 to keep the inside of the air discharge component 206 under negative pressure, bubbles in the chambers 212 are discharged through the gas-liquid separation membrane 204 to the air discharge component 206, which prevents unevenness of the reaction and allows reaction efficiency and detection sensitivity to be raised. Further, by providing the porous membrane 205 between the gas-liquid separation membrane 204 and the chambers 212, a probe fixing region 214 can also be provided on the porous membrane 205 side.

### Embodiment 2

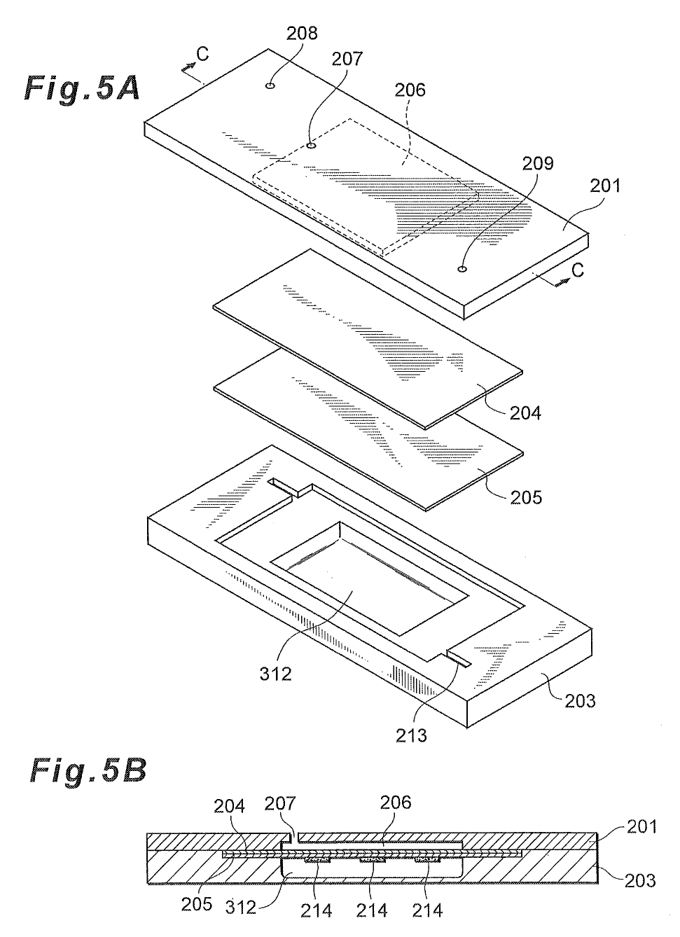

FIG. 5A is an exploded oblique view of a detection cartridge (biological substance detection cartridge) 30 pertaining to Embodiment 2 of the present invention, and FIG. 5B is a cross section along the C-C line in FIG. 5A.

As shown in the drawings, in Embodiment 2, a single reaction vessel 312 is formed instead of the plurality of chambers 212. Also, there is no plate 202, and the gas-liquid separation membrane 204 and the porous membrane 205 are flanked by the plate 201 and the plate 203.

As shown in FIG. 5B, the reaction vessel 312 is formed such that the plate 203 and the porous membrane 205 are superposed over one another, which results in the upper face being covered by the porous membrane 205. Further, by superposing the gas-liquid separation membrane 204 and the plate 201 over this, a configuration is achieved with which any air in the reaction vessel 312 is discharged by the air discharge component 206 through the gas-liquid separation membrane 204. Bubbles generated in the reaction vessel 312 can be removed by keeping the air discharge component 206 at negative pressure.

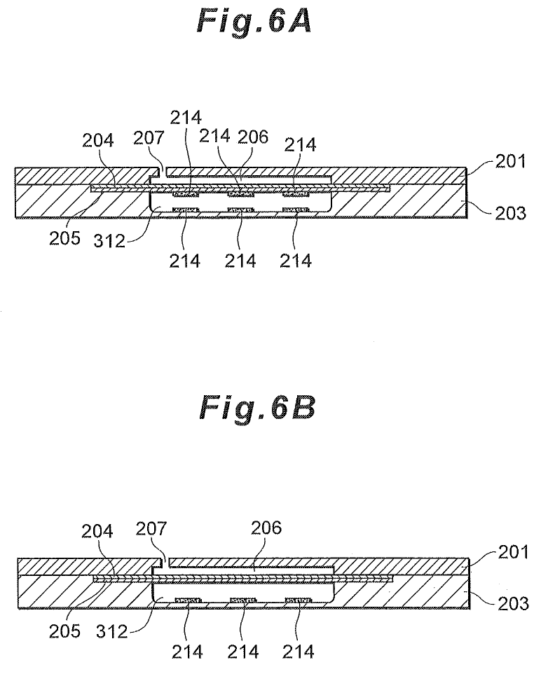

The reaction vessel 312 has a probe fixing region 214 for coating with a probe. The probe fixing region 214 may be provided to the surface of the porous membrane 205 as shown in FIG. 5B. Because the porous membrane 205 has a three-dimensional structure, more probe can be fixed than to the inner walls of the reaction vessel 312, so reaction efficiency and detection sensitivity are improved. Also, the probe fixing region 214 may be provided in a region facing the inner walls of the reaction vessel 312 and to the surface of the porous membrane 205 as shown in FIG. 6A. In this case, the probe coating is applied so that the same type of probe is disposed in the facing region. With the configuration shown in FIG. 6A, the amount of probe in the reaction vessel 312 increases, and the surface area over which the biological substance that is the target comes into contact with the probe is also greater, so reaction efficiency and detection sensitivity are enhanced. Also, the probe fixing region 214 may be provided only to the inner walls of the reaction vessel 312 as shown in FIG. 6B.

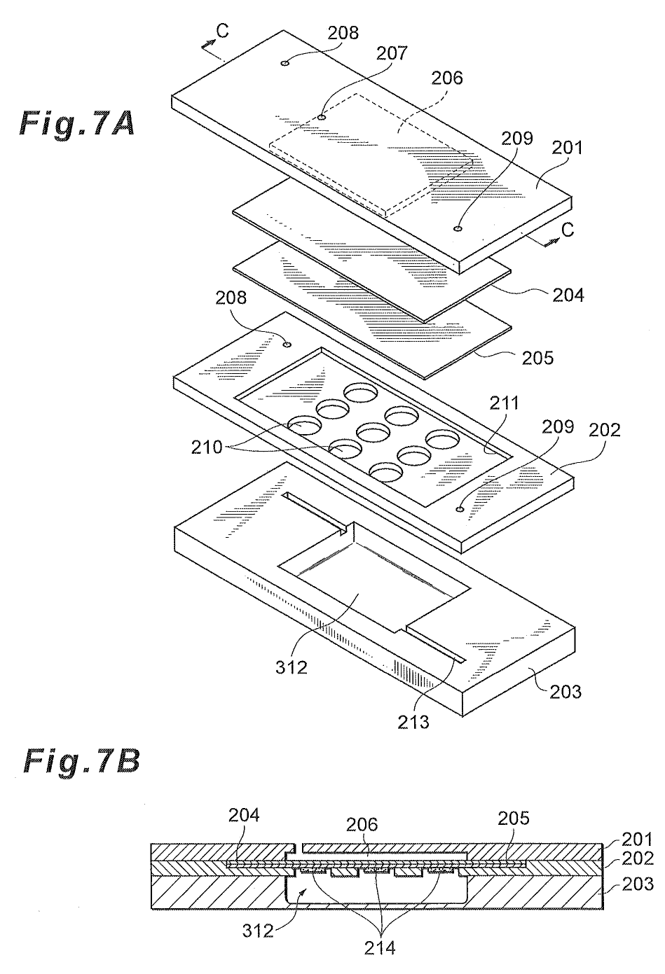

FIG. 7A is an exploded oblique view of a detection cartridge (biological substance detection cartridge) 40 pertaining to a variation of Embodiment 2 of the present invention, and FIG. 7B is a cross section along the C-C line in FIG. 7A. As shown in the drawings, a single reaction vessel 312 is formed on the plate 203, just as with the detection cartridge 30. Also, the detection cartridge 40 is equipped with a plate 202, and the gas-liquid separation membrane 204 and the porous membrane 205 are flanked by the plate 201 and the plate 202.

When the plate 203, the plate 202, and the porous membrane 205 are superposed as shown in FIG. 7B, the reaction vessel 312 is formed such that the upper face is covered by the porous membrane 205. Also, because the plate 202 is sandwiched between the porous membrane 205 and the plate 203, only the porous membrane 205 in the region corresponding to the through-holes 210 provided to the plate 202 is exposed in the reaction vessel 312, so the probe fixing region 214 can be formed in this region. Further, superposing the gas-liquid separation membrane 204 and the plate 201 over the porous membrane 205 affords a configuration in which air in the reaction vessel 312 is discharged through the gas-liquid separation membrane 204 to the air discharge component 206. Bubbles generated in the reaction vessel 312 can be removed by keeping the air discharge component under negative pressure.

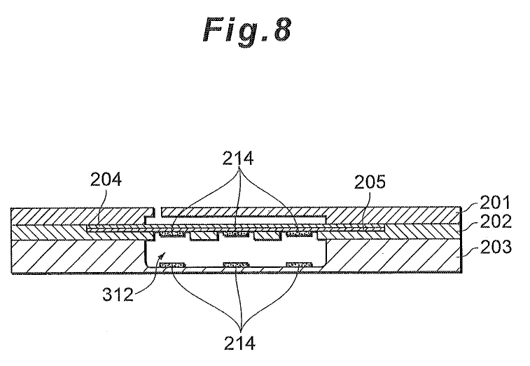

The probe fixing region 214 of the reaction vessel 312 may also be provided just to the surface of the porous membrane as shown in FIG. 7B, or may be provided to the region facing the inner walls of the reaction vessel 312 and the surface of the porous membrane 205 as shown in FIG. 8. In this case, the probe coating is applied so that the same type of probe is disposed in the facing region.

What is claimed is:

1. A biological substance detection cartridge, comprising:
a reaction vessel for reacting a probe with a specific biological substance included in a sample solution, the reaction vessel having a region for fixing the probe for detecting the biological substance;
a porous membrane facing the inside of the reaction vessel;
a gas-liquid separation membrane superposed with the porous membrane; and
a air discharge component which is provided on the opposite side of the gas-liquid separation membrane from the side contacting the porous membrane, and with which the interior can be kept at negative pressure during the reaction between the biological substance and the probe.

2. The biological substance detection cartridge according to claim 1, wherein the reaction vessel comprises:
a plurality of chambers for reacting the biological substance and the probe, each having a region for fixing the probe; and
a channel provided between the plurality of chambers,
the channels being such that the surface area of a cross section perpendicular to a direction in which the sample solution moves is smaller than the cross sectional area of the chamber.

3. The biological substance detection cartridge according to claim 1, the region for fixing the probe being provided to the inner walls of the reaction vessel.

4. The biological substance detection cartridge according to claim 1, the region for fixing the probe being provided over the porous membrane.

5. The biological substance detection cartridge according to claim 1, further comprising a plate having a through-hole corresponding to the region for fixing the probe, between the reaction vessel and the porous membrane.

6. The biological substance detection cartridge according to claim 1, the reaction vessel being formed with a transparent plate.

7. A biological substance detection apparatus for performing biological substance detection by using the biological substance detection cartridge according to claim 1, further comprising a first pump for keeping the air discharge component at negative pressure during the reaction between the biological substance and the probe.

8. The biological substance detection apparatus according to claim 7, comprising a second pump for reciprocally moving the sample solution within the reaction vessel.

9. A biological substance detection method, using the biological substance detection apparatus according to claim 7, comprising:
supplying a sample solution into the reaction vessel, and reacting a specific biological substance included in the sample solution with a probe that is fixed in the reaction vessel and is used to detect the biological substance; and
detecting the biological substance reacted with the probe,
the air discharge component being kept at negative pressure in the reaction step, so that any bubbles in the reaction vessel are discharged through the gas-liquid separation membrane to the outside.

10. The biological substance detection method according to claim 9, in the detection step, detection of the biological substance reacted with the probe being performed by a method using a chemiluminescent substance.
